(19) United States
(12) Patent Application Publication
(10) Pub. No.: US 2008/0086108 A1
(43) Pub. Date: Apr. 10, 2008

(54) Method and apparatus for delivering a drug

(76) Inventors: Michael I. Falkel, Carmel Highlands, CA (US); Matthew J. Stepovich, Santa Cruz, CA (US)

Correspondence Address: DARDI & ASSOCIATES, PLLC, 220 S. 6TH ST., SUITE 2000, U.S. BANK PLAZA, MINNEAPOLIS, MN, 55402, US

(21) Appl. No.: 11/973,007

(22) Filed: Oct. 4, 2007

Related U.S. Application Data
(60) Provisional application No. 60/849,643, filed on Oct. 5, 2006.

(51) Int. Cl. A61M 5/31

(52) U.S. Cl. 604/506; 604/171

(57) ABSTRACT

A device to facilitate the administration of an injection to a patient can comprise a hand piece that comprises of a core and a sheath. The core can comprise of a needle assembly and a thumb piece, and the sheath can enclose a portion of the core, at least in some configurations. In some embodiments, the hand piece can comprise of at least one releasable locking mechanism to enable the sheath to lock onto the core in different positions, particularly a safe position in which the needle assembly is enclosed within the sheath and an exposed position in which the needle is exposed from the sheath. A fluid connection within the core connects the needle assembly with a fluid source outside the core. The user can grip the hand piece by supporting their thumb on the thumb piece and positioning the hand piece between the user's two non-thumb fingers. Thus, the user can employ a traditional one-hand grip corresponding to the use of a manual dental syringe. To unlock the sheath of the hand piece, one of the user's non-thumb fingers can be positioned near the locking mechanism to provide ready release of the locking mechanism such that movement of the sheath relative to the needle assembly can occur to expose the needle for an injection into the patient. After delivery of a drug, the sheath can be moved to enclose the needle for later use or disposal.

### CROSS REFERENCE TO RELATED APPLICATIONS

This application claims the benefit of U.S. Provisional Application No. 60/849,643, filed Oct. 5, 2006 to Falkel et al., entitled “Method and Apparatus for Delivering an Anesthetic,” incorporated herein by reference.

### TECHNICAL FIELD

This invention relates to a method and apparatus for delivering fluid, such as a drug, which in some embodiments can be an anesthetic for dental and medical procedures. More particularly, the invention relates to a hand piece and associated components that facilitates delivery of drugs and the like through a needle.

### BACKGROUND OF THE INVENTION

The handheld dental syringe is a stainless-steel apparatus that has been used for decades in nearly every dental practice. In dental school, all dentists learn to employ a particular one-handed syringe grip, in which the syringe body is held between the index and middle fingers and the thumb is placed in a ring attached to the syringe's plunger. In daily practice, almost every dentist uses this same grip to deliver injections, making it second nature.

### SUMMARY OF THE INVENTION

In some aspects, the invention disclosed herein comprises a pump-based anesthetic delivery system using handpieces that incorporate needle safety mechanisms. In some embodiments, the system allows the dentist to employ the familiar one-handed grip using the hand piece to: (a) release a needle safety mechanism; (b) manipulate the handpiece into position; (c) insert the needle at the injection site; (d) aspirate if necessary; (e) deliver the injection; (f) remove the needle; and (g) re-engage the safety mechanism. The disclosed embodiments, other configurations, objects and advantages of the invention will become more apparent as this description proceeds, reference being made to the accompanying drawings.

In a first aspect, the invention relates to a hand piece for delivering drug. The hand piece comprises a core, a sheath, and a fluid connection within the core connecting the needle assembly with a fluid source outside the core. The core comprises of a thumb piece associated with its proximal portion and a needle assembly in its distal portion. The needle assembly comprises of a needle. The sheath is adapted to move over the core with a first configuration substantially covering the needle portion and a second configuration with the needle extending from the hand piece in a distal direction.

In a further aspect, the invention relates to a method for delivering a drug using a hand piece. The hand piece comprises of a core comprising a needle assembly, a sheath adapted to engage with the core, and a fluid connection within the core connecting the needle assembly with a fluid source outside the core. The method comprises the steps of positioning a hand piece at a site, moving the sheath relative to the core from a first safe position to a second exposed position, and injecting fluid from the fluid source into the site.

In another aspect, the invention relates to a method for handling a hand piece. The hand piece comprises of a core, sheath, and a fluid connection within the core. The core comprises of a needle assembly and a thumb piece. The sheath encloses the core. The fluid connection connects the needle assembly with a fluid source outside the core. The method comprises of the steps of injecting a drug into a patient using the hand piece. The injection is performed while supporting the user's thumb on the thumb piece and with the hand piece between the user's two non-thumb fingers.

### BRIEF DESCRIPTION OF THE DRAWINGS

FIG. 1 is a perspective view of a first embodiment of the invention showing a protective sheath and a handpiece core prior to assembly of the handpiece.

FIG. 2 is a perspective view of the embodiment disclosed in FIG. 1, showing the handpiece assembled and in the safe position.

FIG. 3 is a perspective view of the embodiment disclosed in FIG. 1, showing the handpiece between the safe and exposed positions.

FIG. 4 is a perspective view of the embodiment disclosed in FIG. 1, showing the handpiece locked in the exposed position.

FIG. 5 is a perspective view of the embodiment disclosed in FIG. 1, showing the release of the handpiece from its locked position.

FIG. 6 is a perspective view of the embodiment disclosed in FIG. 1, showing the handpiece between the exposed and safe positions.

FIG. 7 is a perspective view of the embodiment disclosed in FIG. 1, showing the handpiece locked in the safe position.

FIG. 8 is a perspective view of a second embodiment of the invention incorporating finger stays, the drawings showing the components of the handpiece prior to assembly.

FIG. 9 is a perspective view of the embodiment disclosed in FIG. 8, showing the handpiece assembled and in the safe position.

FIG. 10 is a perspective view of the embodiment disclosed in FIG. 8, showing the handpiece between the safe and exposed positions.

FIG. 11 is a perspective view of the embodiment disclosed in FIG. 8, showing the handpiece locked in the exposed position.

FIG. 12 is a perspective view of a third embodiment of the invention incorporating a spring, the drawings showing the components of the handpiece prior to assembly.

FIG. 13 is a perspective view of the embodiment disclosed in FIG. 12, showing the handpiece assembled and in the safe position.

FIG. 14 is a perspective view of the embodiment disclosed in FIG. 12, showing the handpiece between the safe and exposed positions.

FIG. 15 is a perspective view of the embodiment disclosed in FIG. 12, showing the handpiece locked in the exposed position.

FIG. 16 is a perspective view of the embodiment disclosed in FIG. 12, showing the release of the handpiece from the locked and exposed position.

FIG. 17 is a perspective view of the embodiment disclosed in FIG. 12, showing the handpiece between the exposed and safe positions.

FIG. 18 is a perspective view of the embodiment disclosed in FIG. 12, showing the handpiece locked in the safe position.

FIG. 19 shows a system with which the hand piece can be used with, in which the hand piece is depicted in the safe position.

FIG. 20 shows a system with which the hand piece can be used, in which the hand piece is in the exposed position.

FIG. 21 shows a modified cartridge holder that can support the hand piece when the hand piece is not in use.

### DETAILED DESCRIPTION

In general, the apparatuses described herein can be used to deliver a drug or a combination of drugs to a patient. Patients generally can be humans, farm animals, pets or other mammals, although human patients are of particular interest. The drug can be any fluid, which flows appropriately for delivery. Thus, the system can deliver the drug, for example, for ingestion, inhalation or injection into a patient. In embodiments of particular interest, the drug and portions of the flow pathway from the cartridge to the delivery element along with corresponding portions of the apparatus are sterile. An injection can be, for example, subcutaneous, intravenous, intra-arterial, intradermal, or intramuscular, or for injection into bone or other soft tissue. Drugs can be, for example, medicinal/pharmacological compositions, nutrients, such as glucose, volumizing compositions, such as saline, or other beneficial fluids for delivery to the patient. A wide range of drugs are suitable for delivery using the techniques and equipment described herein. Procedures of particular interest include, for example, dental procedures, in which the apparatuses described herein can be used for the delivery of anesthetic into tissue within a patient's mouth.

In general, the cartridges can hold any drug that has characteristics of a fluid. Thus, the drug may be a liquid, flowable powder, a dispersion, an aerosol, a colloid, a gas or the like. In some embodiments, a drug can be a medicinal composition, such as analgesics, anti-inflammatories, antibiotics, antiseptics, anesthetics, vasoconstrictors, combinations thereof or the like. Dental anesthetics include, for example, novocain, lidocaine, prilocalne, mepivicaine and combinations thereof as well as anesthetics mixed with vasoconstrictors, like epinephrine. In some embodiments, a drug in one state, such as a liquid, may be combined with the same or a different drug in a different state, such as a powder or a gas. In some embodiments, the drug comprises a liquid anesthetic, or a mixture of a liquid anesthetic and an alkalinizing substance.

Hand pieces described herein can be used to deliver liquid for medical administration using a pump or other delivery apparatus operably connected to the hand piece. The hand piece and pumps can be used in some embodiments to provide dentists device(s) and method(s) for administering anesthetic to their patients. Furthermore, the device(s) and method(s) can be used by other medical practitioners or non-medical persons in circumstances where fluids are delivered by a hypodermic needle.

The hand pieces described herein can reduce or eliminate needle stick accidents and can facilitate the traditional one-handed grip dentists learned in dental school. The hand piece generally comprises of a core and a sheath that fits over and engages with the core. The core can comprise of a needle assembly in the core's distal portion and a thumb piece in the core's proximal portion. A fluid connection is provided within the core and connects the needle assembly with a fluid source outside the hand piece in a fluid-tight manner. In some embodiments, the fluid connection can run through the thumb piece in a U-shaped path, held in place by interference fit or other means. The fluid connection can also run through another structure in the core. This fluid connection can be a flexible tube that provides the fluid-tight path between the needle assembly and fluid source. The fluid connection (i.e. flexible tube) can connect the needle assembly with multiple fluid sources that can contain the same or different fluid. The fluid source can be in the form of a cartridge.

The sheath is adapted to move relative to a core with the needle covered in at least one configuration and with the needle exposed for use in another configuration. The sheath can move toward or away from the thumb piece to expose the needle assembly from the sheath or enclose the needle assembly within the sheath, respectively. In some embodiments, the sheath can lock with the core in a first safe position and in a second exposed position. The first safe position is where the sheath completely encloses the needle assembly and prevents inadvertent contact with the needle tip. The second exposed position is where the sheath is retracted and the needle tip is exposed to administer fluids to the patient.

In some embodiments, to lock the sheath with the core in the positions, each of the core and the sheath can be provided with at least one or two lock members. To facilitate locking in these two positions, either the sheath or the core can have two such members, and the other of the core or sheath can have one such member. For example, the sheath can be provided with two openings, and the core can be provided with a pogo pin that locks with the openings into the first and second positions.

Alternatively, the sheath can move relative to the core without locking into a position relative to the core or with the option of selectively locking into one of several positions relative to the core. Whether or not the sheath locks relative to the core, the sheath can be provided with a spring to bias the sheath at the safe position such that the user needs to counter the spring to bring the sheath to the exposed position. The spring generally has an outer diameter smaller than the inner diameter of the sheath to allow for its insertion into the sheath. A first spring stop can be provided in the distal portion of the sheath, and an optional second spring stop can be provided in the proximal portion of the sheath. The distal portion of the core can be inserted into the sheath through the sheath's proximal portion. To expose the needle assembly from the sheath, the sheath can be moved relative to the core toward the thumb piece, compressing the spring to the distal portion of the sheath. To enclose the needle assembly within the sheath, the sheath can be moved relative to the core away from the thumb piece, expanding the spring within the sheath. Resilient means other than a spring can be used.

If the sheath can lock selectively at more than two positions, the sheath or the core can be provided with correspondingly more than two locking elements associated with the locking positions. For example, the core can be provided with one pogo pin, and the sheath can be provided with more than two openings. The sheath can be provided with a first opening and a second opening in its proximal portion and a third opening in its central portion with the first opening being closer to the thumb piece. In this configuration, the pogo pin's engagement with the first opening would fully enclose the needle assembly within the sheath, the pogo pin's engagement with the second opening would partially expose the needle assembly from the sheath, and the pogo pin's engagement with the third opening would fully expose the needle assembly from the sheath.

Temporary caps can be attached to the needle assembly, sheath, or both. These caps can serve several significant purposes, including but not limited to preventing needle stick accidents and contamination. These caps can be removed prior to using the hand piece and replaced after using the hand piece.

To facilitate the one-hand grip, a thumb piece can be provided. The thumb piece can be a circular ring, oval, hook, or other suitable shapes. Regardless of the particular thumb piece, in appropriate embodiments, the design provides for the placement of the thumb along the axis prescribed by the needle. Additionally, finger stays can be provided on the sheath to improve grip. These finger stays can be of any size, although the size generally should provide for the interface with a person's fingers. Generally, the one-hand grip on a hand piece is characterized by a thumb piece supporting a thumb and the shaft of the hand piece being positioned between the index finger and the middle finger. The hand piece can also be positioned between other two non-thumb fingers. If the thumb piece is a thumb ring, the thumb can be placed through the thumb ring.

The external and internal configuration of the core and sheath can be a cylindrical, parallelepiped, pyramidal, or combinations thereof. The core need not have the same internal or external configuration as the sheath. The external and internal configurations for either the core or the sheath need not be the same. For example, the core can have a cylindrical external configuration but an internal rectangular configuration, yielding an inner hollow space of rectangular cross-section within the cylindrical structure. A person of ordinary skill in the art would be able to select compatible configurations and dimensions for the core and the sheath, such that the sheath can slide onto the core.

The hand piece can be used with a delivery system. U.S. application Ser. No. 11/820,208, filed Jun. 18, 2007, entitled “Drug Delivery System” describes such a delivery system and is hereby incorporated by reference. However, the versatile hand piece can also be used with other drug delivery systems.

Delivery systems for drugs provide for efficient and convenient delivery of drugs, such as anesthetics, based on versatile designs. The delivery system generally can comprise of a stationary housing, a removable cartridge holder, flexible tubing, and hand piece. The cartridge holder, flexible tubing, and hand piece can be formed as a disposable unit. In some embodiments, the system provides for the easy loading of multiple cartridges or carpules of one or more drugs that can then be selectively delivered to a patient. The cartridges generally can be held by a stationary base unit with a housing. The drugs from the cartridges can be delivered one after another or in combination or in various sequences through a flexible tube to a delivery element, such as a hand piece with a hypodermic needle as described herein. In additional or alternative embodiments, the base unit comprises one or more heating or cooling elements so that the cartridges, the fluid within the cartridges and/or other system components can be heated or cooled or both before delivery to the patient. A motor can be used to move pistons or plungers to drive the drug from the cartridges for delivery to the patient. The systems and methods are particularly useful for anesthetizing dental patients.

The hypodermic syringe has been an integral part of the medical and dental professions for long periods of time. Other delivery systems have been developed in the heath care industry for delivery of a range of drugs (chemical, biologic or other substances used in health care), such delivery systems including, for example, nebulizers, intravenous bottles, catheters and the like. In dentistry, one widely-used delivery system is comprised of a disposable pre-filled anesthetic cartridge that is loaded into the body of a hand held hypodermic syringe. The syringe is then used to inject the anesthetic from the cartridge into the patient's tissue.

In order to establish a fluid connection between the needle assembly and a fluid source, flexible tubing within the core can extend from the needle assembly through the thumb piece in a U-shaped path to a fluid source outside the hand piece. The flexible tubing may or may not extend through the thumb piece. The first end of the flexible tubing connects with the needle assembly or a fluid channel through the core of the hand piece in fluid communication with the fluid assembly, and the second end of the flexible tubing connects with the cartridge holder or other component of the fluid delivery system.

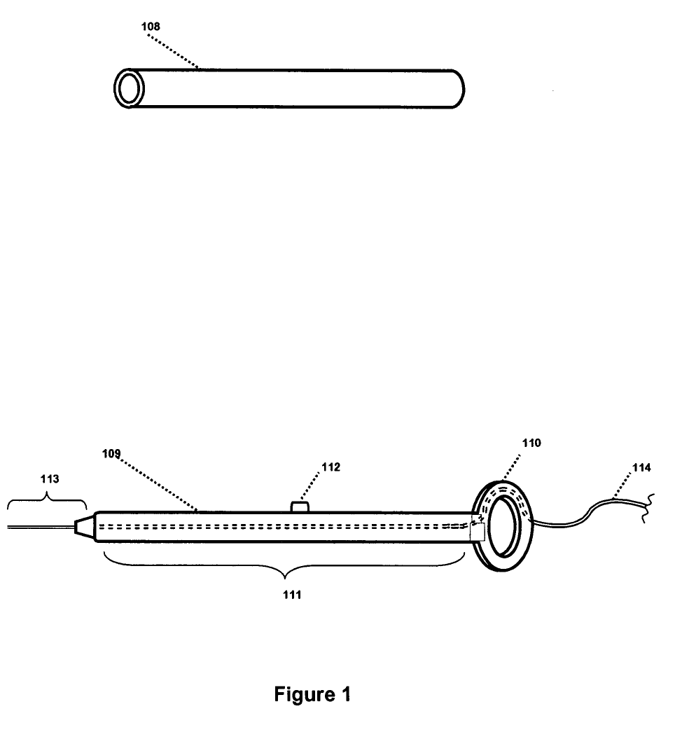

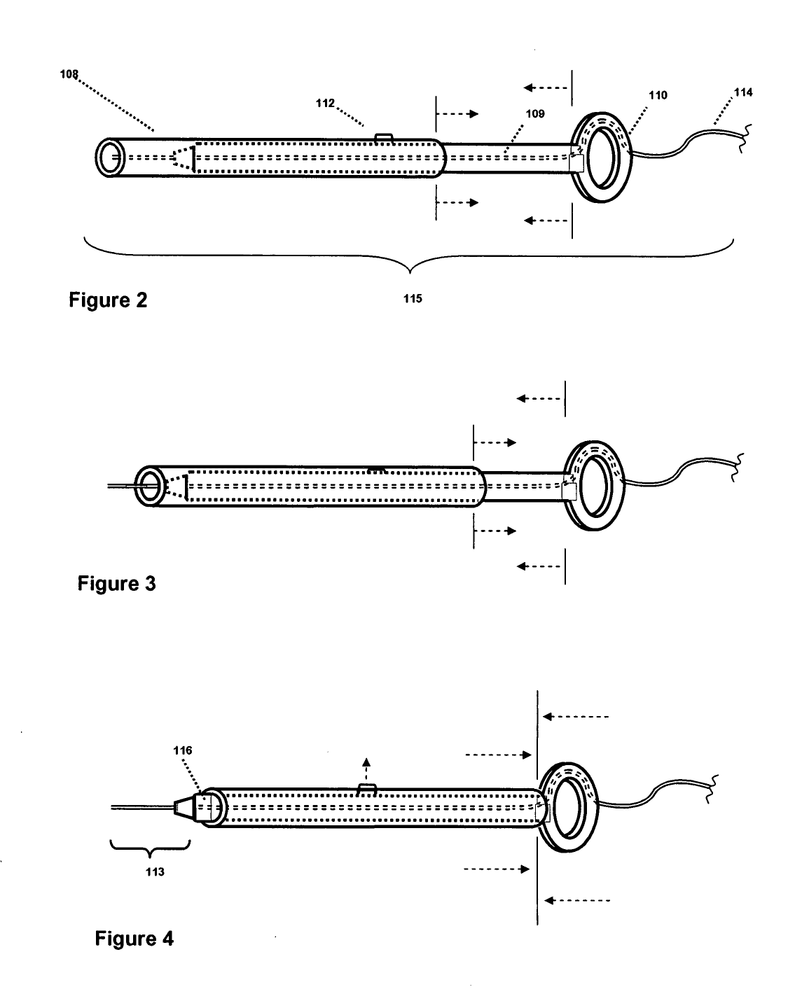

FIG. 1 shows a protective sheath 108 and hand piece core 109 prior to their attachment to each other during the assembly of hand piece 115, which is described in FIG. 2 below. Sheath 108 is a hollow tube with a proximal portion (right) and a distal portion (left). Core 109 has a proximal portion (right) and a distal portion (left). Core 109 is comprised of thumb piece 10 located at the core's proximal portion, a lock element 112 located at the core intermediate portion, and needle assembly 113 located at the core's distal portion. Flexible tube 114 connects needle assembly 113 to an anesthetic source (not shown). Flexible tube 114 enters core 109 at the proximal portion of ring 110 and runs along a U-shaped channel in ring 110 through intermediate portion 111 (the path of the channel is indicated by the dashed line) to needle assembly 113. Flexible tube 114 establishes a fluid tight path between the anesthetic source and the tip of the hypodermic needle.

FIG. 2 is a perspective drawing of an embodiment of the invention in FIG. 1, showing sheath 108 and core 109 assembled as hand piece 115, which is shown in the “safe position”. During the assembly of hand piece 115, the distal portion of core 109 is fit telescopically into the opening (not visible in this perspective) at the proximal portion of sheath 108, sliding core 109 laterally into sheath 108 to a point where the tip of needle assembly 113 is near the opening at the distal portion of sheath 108. In this position, the tip of the hypodermic needle remains enclosed in and protected by sheath 108. When core 109 reaches this point, pogo pin 112 springs up through a first vertical opening (not visible in this perspective) in sheath 108, thereby preventing sheath 108 and core 109 from sliding further laterally relative to each other.

With handpiece 115 in the safe position, the dentist prepares to use it by placing his or her thumb in ring 110, and placing sheath 108 between the index and middle fingers—approximately at the point where pogo pin 112 extends above sheath 108. In order to begin using the handpiece for an injection, the dentist will release the handpiece from the safe position by depressing pogo pin 112, such as using the side of the his or her index finger, releasing sheath 108 and core 109 to move laterally toward each other (indicated by the arrows) as the dentist draws his or her thumb and fingers toward each other.

FIG. 3 is a perspective drawing of the embodiment disclosed in FIG. 2, showing hand piece 115 approximately half way between the safe and exposed positions. As the dentist continues to draw his or her thumb and fingers toward each other, sheath 108 and core 109 continue to move laterally toward each other as indicated. The top of pogo pin 112 remains depressed below the surface of sheath 108, as shown, during the transition.

FIG. 4 is a perspective drawing of the embodiment disclosed in FIG. 2, showing the handpiece in the exposed configuration. The dentist has drawn his or her thumb and fingers together until the proximal portion of sheath 108 made contact with ring 110. Pogo pin 112 springs upward through a second vertical opening (not visible in this perspective) in sheath 108 to lock sheath 108 and core 109 relative to each other in the exposed position, with needle assembly 113 extended completely beyond the distal portion of sheath 108, a portion of core 109 also extending beyond sheath 108, as shown, exposing aspiration window 116.

Using the procedures set forth in the discussion accompanying FIGS. 2-4, the dentist may move the needle from the safe position into the exposed position at any point prior to beginning the injection, however a suitable method is to wait until the distal portion of the sheath protecting the needle is near the site where anesthetic is to be delivered, thus preventing a needle stick while the dentist maneuvers the needle and handpiece into position for the injection. In the safe position, the sheath also screens the needle from the patient's view.

In some embodiments, prior to positioning handpiece 115 for an injection, the dentist has primed the system by beginning the flow of anesthetic from the anesthetic source and maintaining the flow long enough to clear any bubbles from the system and fill the fluid path with anesthetic. With the system primed and the hypodermic needle in the exposed position, the dentist restarts the flow of anesthetic and inserts the needle into the patient's tissue at the injection site. The dentist advances the needle in the patient's tissue to the location where the dentist intends to administer the bulk of the anesthetic, at which point the dentist may elect to aspirate, or reverse the flow of anesthetic in the system for a short time sufficient to create negative pressure in the system and to draw a small amount of fluid from the patient's tissue through the hypodermic needle and into fluid path at aspirating window 116. The dentist inspects the aspirating window for presence of blood in the fluid. The dentist may then elect to proceed with the injection, if appropriate, by re-initiating the flow of anesthetic from the anesthetic source, continuing until the dentist determines that sufficient anesthetic has been deposited into the patient's tissue. The dentist may then remove the needle from the first injection site and proceed to one or more other sites, repeating the above described processes as necessary, or end the injection process and remove the handpiece from the patient's mouth. Immediately upon finishing the injection or removing the handpiece, the dentist may move the needle from the exposed position to the safe position using the procedures described immediately below.

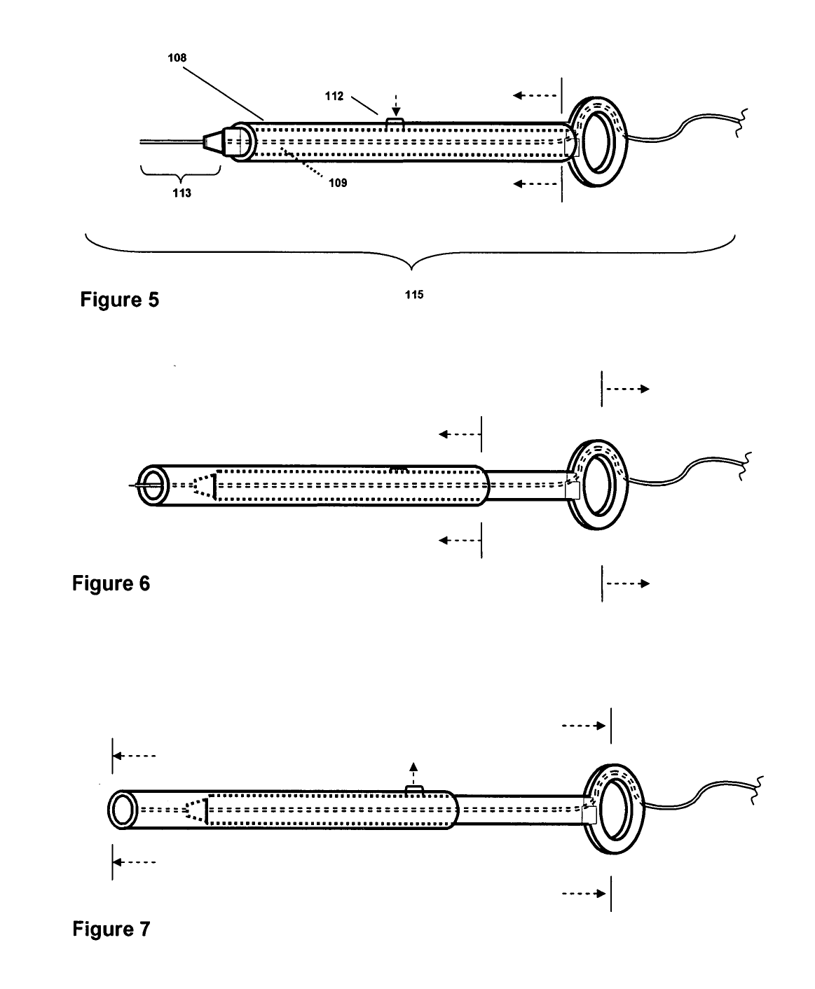

FIG. 5 is a perspective drawing of the embodiment disclosed in FIG. 2, showing the release of handpiece 115 from the exposed position. The dentist depresses pogo pin 112 as indicated, releasing sheath 108 and core 109 to be moved toward the safe position as the dentist moves his or her thumb and fingers away from each other.

FIG. 6 is a perspective drawing of the embodiment disclosed in FIG. 2, showing the handpiece moving from the exposed position toward the safe position. Needle assembly 113 still protrudes partly beyond the distal portion of sheath 108. Pogo pin 112 remains depressed and slides along the interior surface of sheath 108.

FIG. 7 is a perspective drawing of the embodiment disclosed in FIG. 2 showing the handpiece moved from the exposed position fully toward the safe position and locked in the safe position by pogo pin 112, which has again sprung up through the first vertical opening in sheath 108. At the conclusion of this process, handpiece 115 has been returned to the safe position.

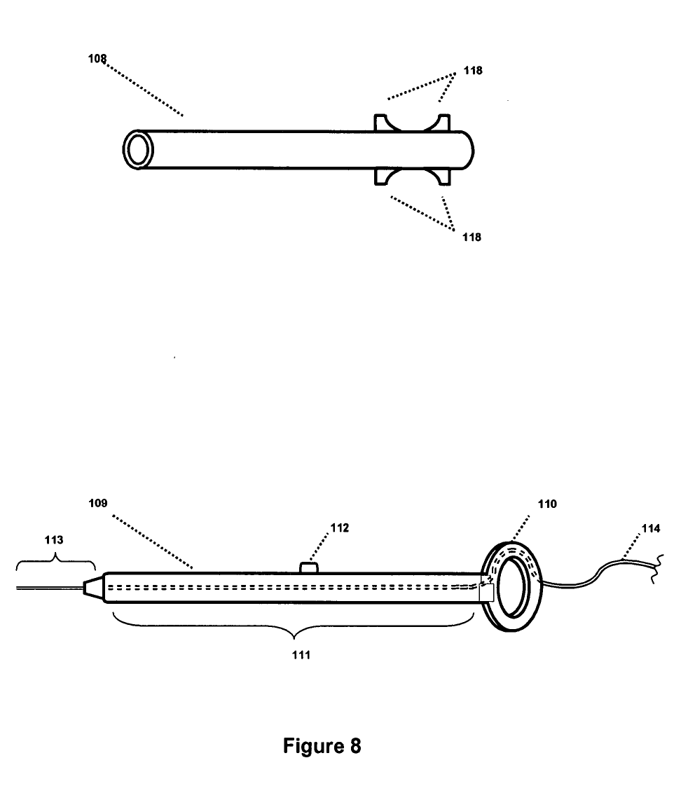

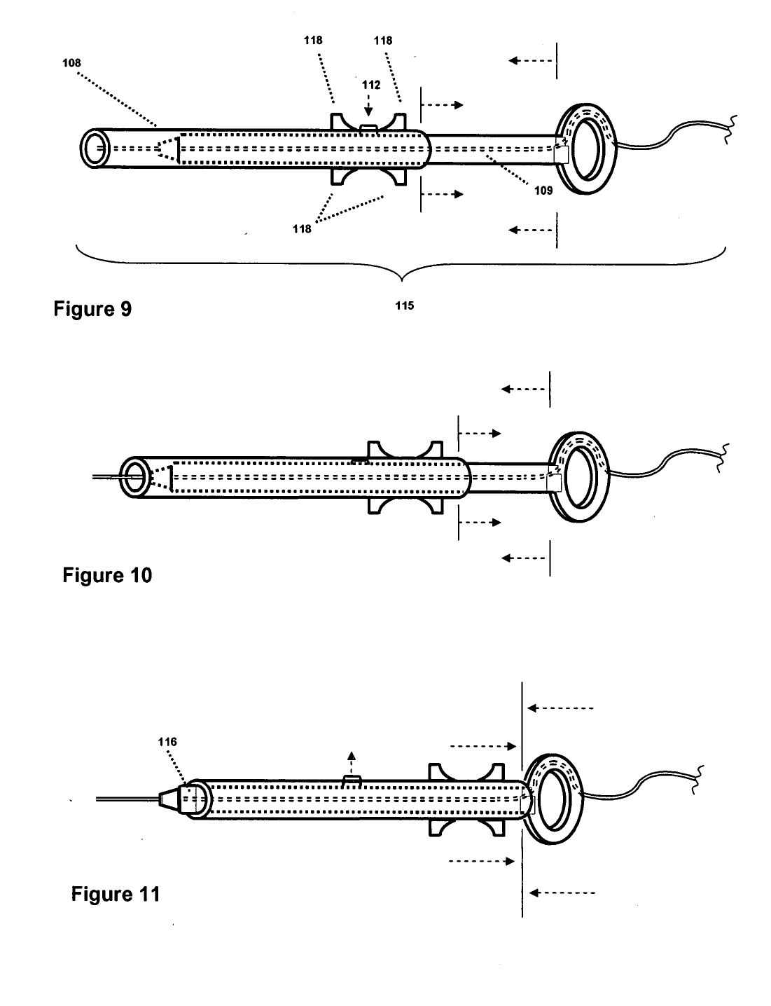

FIG. 8 is a perspective drawing of a second embodiment of the invention with the same elements described in FIG. 1, except that in this embodiment finger stays 118 have been added to improve the dentist's grip on sheath 108 between his or her middle fingers. This view shows sheath 108 and core 109 prior to their assembly into handpiece 115, as described in FIG. 9.

FIG. 9 is a perspective drawing of the embodiment disclosed in FIG. 8, showing sheath 108 (including finger stays 118) and core 109 as they appear assembled as handpiece 115, which is shown in the safe position. The vertical arrow indicates the direction of pressure applied by the dentist on pogo pin 112 to release handpiece 115 from the safe position. This release is accomplished in the same manner as the procedure set forth in the description of FIG. 2.

FIG. 10 is a perspective drawing of the embodiment disclosed in FIG. 9, showing hand piece 115 moving from the exposed position toward the safe position. This embodiment functions in the same manner as set forth in the description accompanying FIG. 3, except that finger stays 118 provide an improved grip for the dentist.

FIG. 11 is a perspective drawing of the embodiment disclosed in FIG. 9, showing hand piece 115 moved from the exposed position fully toward the safe position and locked in the safe position by pogo pin 112, which has sprung up through the second vertical opening (not visible in this perspective) in sheath 108. This embodiment functions in the manner as set forth in the description accompanying FIG. 4, except that the finger stays provide and improved grip for the dentist.

After the injection is complete, handpiece 115 is moved from the exposed position back into the safe position using the same procedure set forth in the description accompanying in FIGS. 5-7, the only difference being that the dentist may use finger stays 118 for an improved grip on handpiece 115.

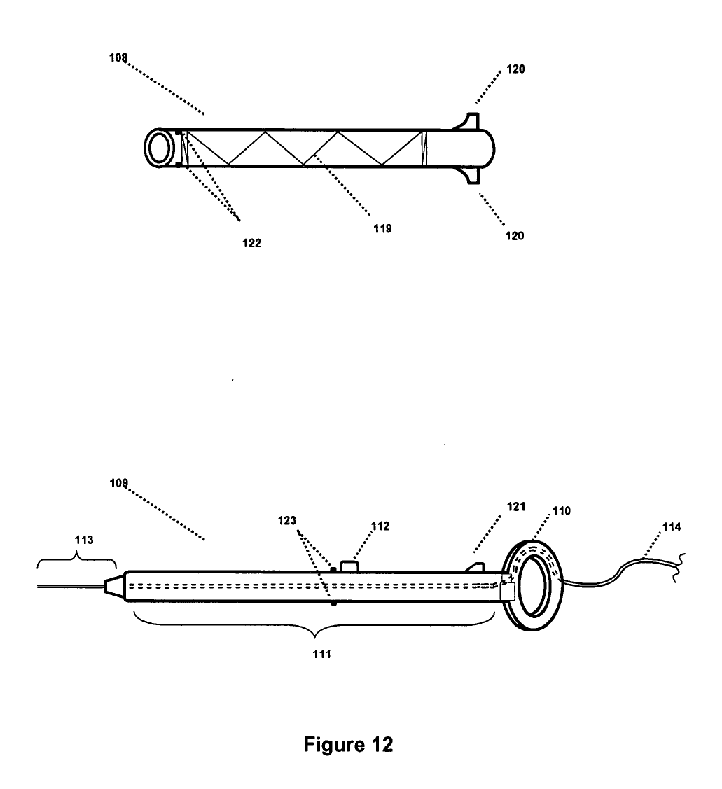

FIG. 12 is a perspective drawing of the third embodiment of the invention with each of the elements shown in FIG. 1, but with the addition of spring 119, finger stays 120, second pogo pin 121, spring stops 122, and spring stops 123. This embodiment has just a single vertical opening (not visible in this perspective) near the proximal portion of sheath 108. Sheath 108 is a hollow tube with a proximal portion (right) and a distal portion (left). Spring 119 has a proximal portion (right) and a distal portion (left). The view in FIG. 12 shows sheath 108 and core 109 prior to their assembly into handpiece 115. The view in FIG. 12 shows spring 109 assembled with core 108 as described immediately below.

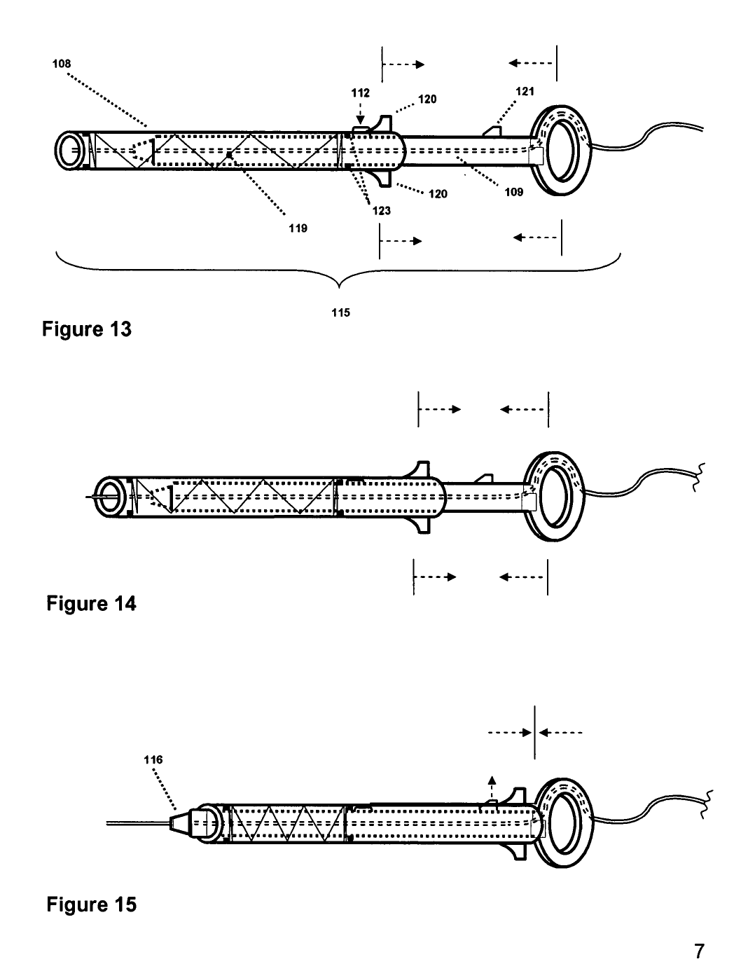

The outside diameter of spring 119 is slightly smaller than the inside diameter of sheath 108, allowing spring 119 to be inserted into sheath 108 by sliding the distal portion of spring 119 into the proximal portion of sheath 108, and continuing to slide spring 119 laterally along the interior surface of sheath 108 until spring 119 reaches spring stops 122, as shown. With spring 119 and sheath 108 assembled in this manner, the proximal portion of spring 119 may be forced laterally toward the distal portion of spring 119, which will compress spring 119, as more fully discussed in the descriptions accompanying FIGS. 13-15.

Hand piece core 109 has a proximal portion (right) and a distal portion (left). The outside diameter of core 109 is slightly smaller than the inside diameter of spring 119, allowing core 109 to be inserted into the interior of spring 119 during the assembly of handpiece 115 as set forth below in the discussion accompanying FIG. 13. Core 109 is comprised of thumb ring 110 located at the core's proximal portion, intermediate portion 111 including pogo pins 112 and 121, spring stops 123, and needle assembly 113 located at the core's distal portion. Flexible tube 114 connects to an anesthetic source (not shown), enters core 109 at the proximal portion of ring 110, runs along a U-shaped channel (as indicated by the dashed line) and connects to needle assembly 113. Flexible tube 114 establishes a fluid tight path between the anesthetic source and the tip of the hypodermic needle.

FIG. 13 is a perspective drawing of the embodiment disclosed in FIG. 12, showing sheath 108 and core 109 assembled into handpiece 115, which is shown in the safe position. During the assembly of handpiece 115, the distal portion of core 109 is fit telescopically into the proximal portion of sheath 108 (which already has spring 119 attached), sliding core 109 laterally into the interior opening of spring 119, to a point where the tip of needle assembly 113 is near the distal opening of sheath 108. The proximal portion of spring 119 makes contact with spring stops 123, which prevent the proximal portion of spring 119 from sliding any further toward the proximal portion of core 109. When core 109 has been inserted to this point, pogo pin 112 springs up through a vertical opening (not visible in this perspective) in sheath 108, thereby locking the handpiece in the safe position and preventing sheath 108 and core 109 from sliding laterally in either direction.

With handpiece 115 in the safe position, the dentist prepares to use it by placing his or her thumb in ring 110, and placing sheath 108 between the index and middle fingers, with the index and middle fingers pulling slightly against finger stays 120. These three points of contact provide the dentist a familiar one-handed grip on the handpiece. In order to begin using the handpiece for an injection, the dentist will release the sheath from the safe position by depressing pogo pin 112 preferably with the side of his or her index finger, allowing sheath 108 and core 109 to be drawn laterally toward each other (indicated by the lateral arrows) as the dentist draws his or her thumb and fingers together. The lateral motion of sheath 108 toward the proximal portion of core 109 will press spring stops 123 against the proximal portion of spring 119 and springs stops 122 against the distal portion of spring 119, causing spring 119 to be compressed as sheath 108 is drawn further toward the proximal portion of core 109. Among other things, the compression will provide resistance and improved tactile control over the movement of the sheath from the safe position to the exposed position, and vice verse.

FIG. 14 is a perspective drawing of the embodiment disclosed in FIG. 13, showing the handpiece approximately half way between the safe and the exposed positions. As the dentist continues to draw his or her thumb and fingers toward each other (pulling against finger stays 120) sheath 108 and core 109 will continue to move laterally toward each other as indicated, further compressing spring 119. In this view, the top of pogo pin 112 is depressed below the surface of sheath 108, as shown.

FIG. 15 is a perspective drawing of the embodiment disclosed in FIG. 13, showing the handpiece in the “exposed” configuration or position. The dentist has continued to draw his or her thumb and fingers together until sheath 108 slides over pogo pin 121 (compressing it) after which the proximal portion of sheath 108 continues laterally until it makes contact with ring 110. With the handpiece in this fully exposed position, pogo pin 121 is centered in the vertical opening in sheath 108, allowing pogo pin 121 to spring vertically into the position shown in FIG. 15, where it locks handpiece 115 in the exposed position. Needle assembly 113 is extended completely beyond the distal portion of sheath 108, with a portion of core 109 also extending beyond sheath 108, as shown, exposing aspiration window 116 nearest needle assembly 113. Spring 119 is further compressed.

The dentist may move the needle from the safe position into the exposed position, as described above, at any point prior to beginning the injection, however a preferred method is to wait until the distal portion of the sheath protecting the needle is near the site where anesthetic is to be delivered, thus preventing a needle stick while the dentist maneuvers the needle and handpiece into position for the injection. In the safe configuration, the needle is also screened from the patient's view.

Preferably prior to positioning handpiece 115 for an injection, the dentist will have primed the system by beginning the flow of anesthetic from the anesthetic source and maintaining the flow long enough to clear any bubbles from the system and fill the fluid path with anesthetic. With the system primed and the hypodermic needle in the exposed position, the dentist restarts the flow of anesthetic and inserts the needle into the patient's tissue at the injection site. The dentist advances the needle in the patient's tissue to the location where the dentist intends to administer the bulk of the anesthetic, at which point the dentist may elect to aspirate, or reverse the flow of anesthetic in the system for a short time sufficient to create negative pressure in the system and to draw a small amount of fluid from the patient's tissue through the hypodermic needle and into fluid path at aspirating window 116. The dentist inspects the aspirating window for presence of blood in the fluid. The dentist may then elect to proceed with the injection, if appropriate, by re-initiating the flow of anesthetic from the anesthetic source, continuing until the dentist determines that sufficient anesthetic has been deposited into the patient's tissue. The dentist may then remove the needle from the first injection site and proceed to one or more other sites, repeating the above described processes as necessary, or end the injection process and remove the handpiece from the patient's mouth. Immediately upon finishing the injection or removing the handpiece, the dentist may move the needle from the exposed position to the safe position using the procedures described below.

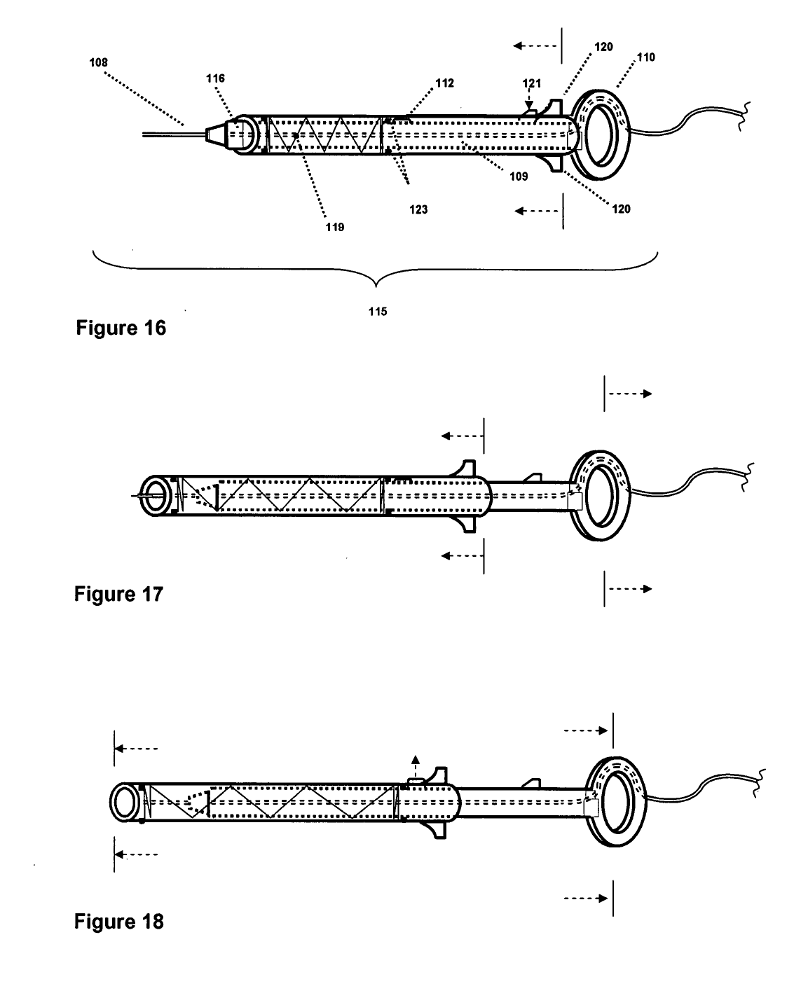

FIG. 16 is a perspective view of the embodiment disclosed in FIG. 15, showing the process of releasing handpiece 115 from the exposed position. While maintaining the one-handed grip on the handpiece, the dentist places downward pressure on pogo pin 121 with the side of his or her index finger, releasing sheath 108 and core 109 to move laterally relative to each other. Spring 119 will then be free to expand, which will bias sheath 108 distally, toward the safe position. The dentist can control the rate at which sheath 108 moves in response to pressure from the expanding spring, by controlling the distance he or she allows the thumb and fingers to move apart, the fingers resisting the pull of finger stays 120.

FIG. 17 is a perspective view of the embodiment disclosed in FIG. 15, showing hand piece 115 moving between the exposed and safe position. Spring 119 continues to expand and bias sheath 108 toward the safe position, control over the rate being maintained by the dentist's fingers and thumb.

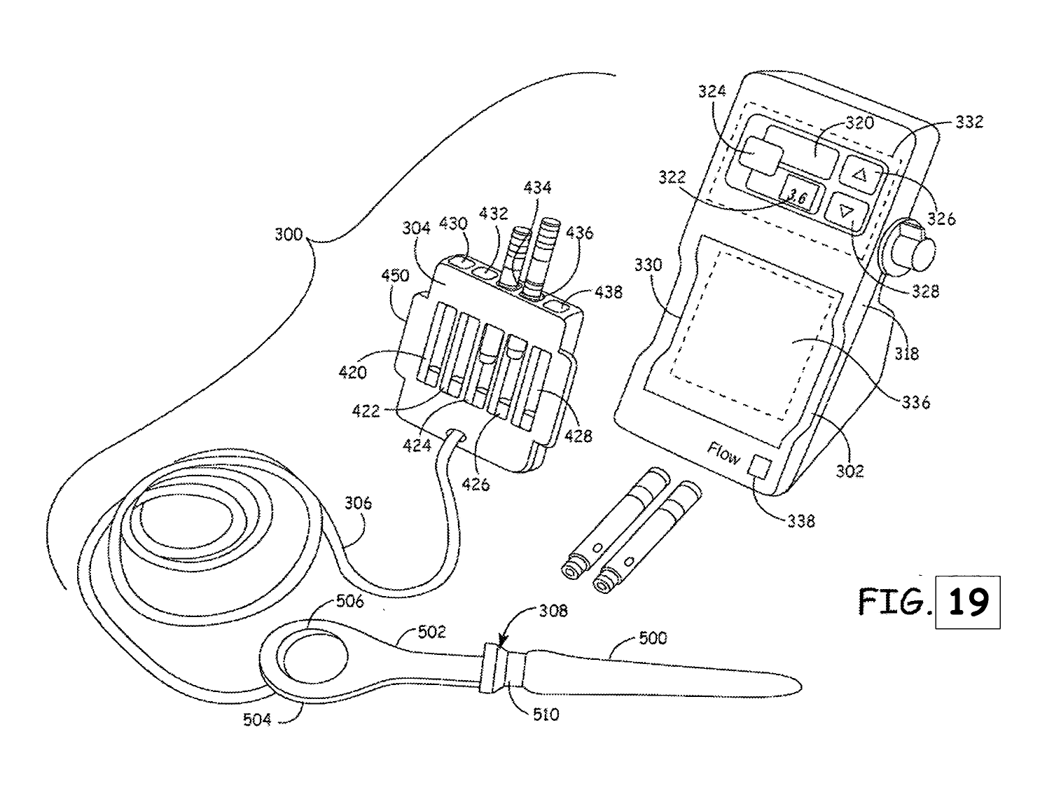

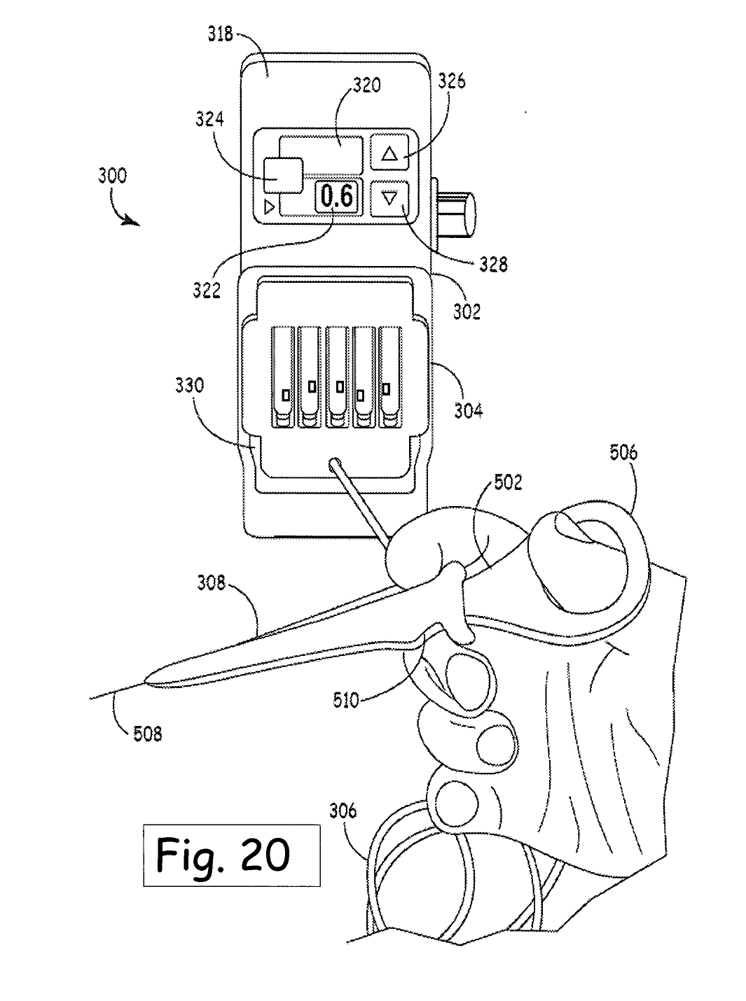

FIG. 18 is a perspective drawing of the embodiment disclosed in FIG. 15 showing hand piece 115 moved from the exposed position fully toward the safe position. Pogo pin 112 has sprung up through the vertical opening (not visible in this perspective) in sheath 108, locking hand piece 115 back into the safe position.

The hand piece can be incorporated into a delivery system. Referring to FIG. 19, delivery system 300 comprises a stationary housing 302, a removable cartridge holder 304, flexible tubing 306 and hand piece 308. The cartridge holder 304, flexible tubing 306, and hand piece 308 as a combination can be a disposable unit such that any portion of the system that may be exposed to the blood of one patient is disposed prior to being used on a subsequent patient. Hand piece 308 has a fluid connection with flexible tubing 306. Hand piece 308 comprises a sheath 500 and a core 502. Sheath 500 moves relative to the core 502 with a spring to control this movement with the unbiased position being a closed configuration to reduce the risk of accidental needle pricks. The core 502 comprises connection 504 to connect to flexible tubing 306. The core 502 further comprises thumb piece 506 and needle assembly (not visible from within the sheath) with a fluid connection between tubing connection 504 and needle assembly. The sheath 500 comprises grip 510.

Also shown in FIG. 19, the stationary housing 302 comprises housing 318, displays 320, 322, input pads 324, 326, 328, docking section 330, controller 332, drive unit 334, heater 336, and heater on-off switch 338. Display 320 comprises three separate elements that can light to indicate flow at one of three speeds, slow, medium, or fast, and display 322 comprises a two digit display to depict the dose the practitioner elects to deliver. After delivery has begun, display 322 may transition to a mode where it displays the running amount of drug that has actually been delivered to the patient. Docking section 330 is configured to accept removable cartridge holder 304. Controller 332 can comprise a microprocessor and/or other appropriate logic control circuits.

FIG. 20 depicts hand piece 308 in a delivery configuration with needle 508 exposed for use. The overall design of hand piece 308 mimics the grip and hand alignment of a conventional dental syringe so that an experienced dentist can efficiently transition to the use of hand piece 308 with a desirable comfort level, and can comfortably move shield element 500 from the safe position, with the needle covered, to the ready position (as shown in FIG. 20) and back to the safe position when the injection is finished.

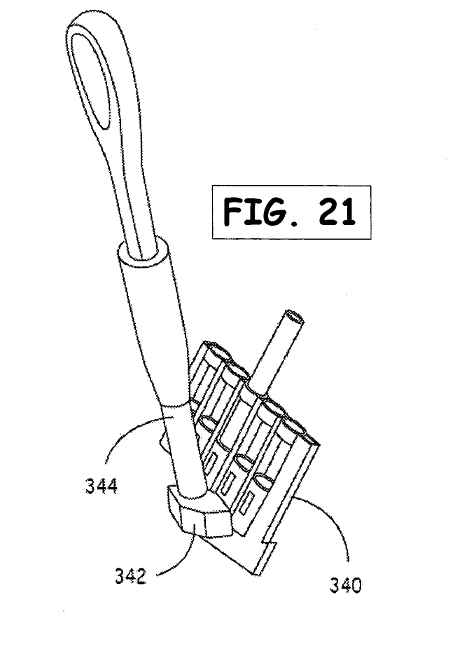

FIG. 21 depicts a modified cartridge holder 340 that has a hand piece support comprising a first element 342 and a second element 344 such that hand piece 308 can be conveniently supported when not in use. It is convenient to have a hand piece support on the cartridge holder since contact with the hand piece after use can result in contamination with bodily fluid and since the cartridge holder can be disposed following the procedure. Other designs of the hand piece support can be used as desired. Cartridge holder element 342 may comprise or be connected to a heating element that delivers heat to the hand piece.

The apparatus can comprise a manifold, and the cartridge holder can have positions for a plurality of cartridges such that the plurality of cartridges all feed into the flexible tubing through the manifold. In this way, fluids from a plurality of cartridges can be sequentially and/or simultaneously delivered through the tubing. The cartridge holder generally comprises a transfer tube that interfaces with a cartridge to establish a fluid connection between the transfer tube and the contents of the cartridge. The delivery element can be any suitable device to deliver a drug to a patient generally in sterile condition. A suitable delivery element can be for example, a hand piece with a hypodermic needle, an infuser, a nebulizer, a catheter or other apparatuses or combination of apparatuses that deliver the drugs from the flexible tubing to the patient.

If there is a manifold, a plurality of transfer tubes lead to the manifold. If a cartridge is loaded onto each transfer tube, then the contents of the cartridges are combined in the manifold, and the system generally is fluid tight from the cartridge(s) through the delivery element. However, in some embodiments, the system remains fluid tight even with one or more slots in the cartridge holder “open” or not loaded with a cartridge. This ability provides the system the flexibility to function fully or partially loaded.

If there is a manifold, the contents of the cartridges can be heated or cooled or both by directly heating or cooling the manifold instead of directly heating or cooling the cartridges. This temperature control approach avoids damaging or melting cartridges that might be plastic and helps to control more directly the temperature of the flowing drug.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein.

What is claimed is:

1. A hand piece for delivering a drug comprising:
a core having a proximal portion and a distal portion, wherein the core comprises:
a thumb piece associated with the proximal portion, and
a needle assembly in the distal portion comprising a needle;
a sheath adapted to move over the core with a first configuration substantially covering the needle portion and a second configuration with the needle extending from the hand piece in a distal direction;
a fluid connection within the core connecting the needle assembly with a fluid conduit extending from the core.

2. The hand piece of claim 1 wherein the sheath is adapted to lock selectively with the core at a first safe position and, alternatively, at a second exposed position, the needle assembly being enclosed by the sheath in the first safe position and being exposed from the sheath in the second exposed position.

3. The hand piece of claim 2,
wherein the core comprises of at least one core lock element, and
wherein the sheath comprises of at least one sheath lock element.

4. The hand piece of claim 3,
wherein the core lock element comprises of a protrusion; and
wherein the sheath lock element comprises of a first opening and a second opening, the protrusion being snapped into the first opening to lock the sheath into the first safe position or the protrusion being snapped into the second opening to lock the sheath into the second exposed position.

5. The hand piece of claim 3,
wherein the core lock element comprises of an opening; and
wherein the sheath lock element comprises of a first protrusion and a second protrusion, the first protrusion being snapped into the opening to lock the sheath into the first safe position or the second protrusion being snapped into the opening to lock the sheath into the second exposed position.

6. The hand piece of claim 3,
wherein the core lock element comprises of either a ridge; and
wherein the sheath lock element comprises of a first depression and a second depression, the ridge being snapped into the first depression to lock the sheath into the first safe position or the ridge being snapped into the second depression to lock the sheath into the second exposed position.

7. The hand piece of claim 1, wherein the fluid connection runs along the core from the needle assembly out through the thumb piece to the fluid source outside the core.

8. The hand piece of claim 1, wherein the thumb piece comprises a thumb ring through which a thumb is placed.

9. The hand piece of claim 6, wherein the fluid connection runs along the core from the needle assembly through a U-shaped path in the thumb ring to the fluid source outside the core.

10. The hand piece of claim 1, wherein the sheath comprises finger stays, the finger stays comprising a curved surface to support a finger.

11. The hand piece of claim 3, wherein the sheath comprises finger stays around a sheath lock element, the finger stays comprising a curved surface to improve grip on the sheath.

12. The hand piece of claim 1, wherein the sheath comprises a resilient structure, the resilient structure comprising a first stop and a second stop.

13. A method for delivering a drug using a hand piece comprising a core comprising a needle assembly, a sheath adapted to engage with the core, and a fluid connection within the core connecting the needle assembly with a fluid source outside the core, the method comprising:
moving the sheath relative to the needle assembly from a safe position to a ready position, wherein the needle assembly is substantially enclosed within the sheath in the safe position and the needle assembly is exposed from the sheath in the ready position; and
injecting fluid from the fluid source into the tissue of a patient.

14. The method of claim 13, further comprising positioning the hand piece at the site of injection prior to the step of moving the sheath from a safe position to a ready position.

15. The method of claim 13, further comprising starting the flow of fluid prior to the step of injecting fluid from the fluid source into the site is performed.

16. The method of claim 13,
wherein the core further comprises an aspirating window, and
wherein the step of injecting fluid comprises drawing fluid from the site into a fluid path at the aspirating window.

17. The method of claim 16, further comprising inspecting the aspirating window for blood.

18. The method of claim 13, further comprising enclosing the needle assembly within the sheath.

19. The method of claim 13, further comprising positioning the hand piece at a second site.

20. The method of claim 13, further comprising cooling the fluid prior to injecting the fluid.

21. A method for handling a hand piece in a tripod grip comprising a core comprising a needle assembly and a thumb piece, a sheath enclosing the core, and a fluid connection within the core connecting the needle assembly with a fluid source outside the core, the method comprising:
injecting a drug into a patient using the hand piece, the injection being performed while supporting the user's thumb on the thumb piece and with the hand piece between the user's two non-thumb fingers.

22. The method of claim 21, wherein the hand piece comprises of at least one releasable locking mechanism for locking the sheath with the core in at least one position.

23. The method of claim 21, further comprising drawing the user's non-thumb fingers toward the user's thumb to expose the needle assembly from the sheath.

24. The method of claim 21, further comprising drawing the user's non-thumb fingers away from the user's thumb to substantially enclose the needle assembly within the sheath.
